(19) United States
(12) Patent Application Publication
(10) Pub. No.: US 2008/0033539 A1
(43) Pub. Date: Feb. 7, 2008

(54) STENT HAVING GENISTEIN-CONTAINING COATING OR CAVITY FILLING

(75) Inventors: Katrin Sternberg, Rostock (DE); Klaus-Peter Schmitz, Warnemuende (DE); Detlef Behrend, Rostock (DE); Gerhard Hennighausen, Lambrechtshagen (DE); Claus Martini, Zurich (CH); Claus Harder, Uttenreuth (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/834,469

(22) Filed: Aug. 6, 2007

(30) Foreign Application Priority Data
Aug. 7, 2006 (DE) 10 2006 038 241.2

(51) Int. Cl. A61F 2/06

(52) U.S. Cl. 623/1.46

(57) ABSTRACT

A stent made of a biocorrodible metallic material and having a coating or cavity filling which contains genistein.

### PRIORITY CLAIM

This patent application claims priority to German Patent Application No. 10 2006 038 241.2, filed Aug. 7, 2007, the disclosure of which is incorporated herein by reference in its entirety.

### FIELD

The present disclosure relates to a stent made of a biocorrodible metallic material having a coating or cavity filling containing an active ingredient, and a use of genistein.

### BACKGROUND

The implantation of stents has established itself as one of the most effective therapeutic measures in the treatment of vascular illnesses. Stents have the purpose of assuming a support function in the hollow organs of a patient. Stents of typical construction have filigree support structure made of metallic struts for this purpose, which is first provided in a compressed form for introduction into the body and is expanded at the location of application. One of the main areas of application of such stents is permanently or temporarily expanding and keeping open vascular constrictions, in particular constrictions (stenoses) of the coronary vessels. In addition, for example, aneurysm stents are also known, which are used to support damaged vascular walls.

Stents have a peripheral wall of sufficient carrying force to keep the constricted vessel open to the desired degree and a tubular main body through which the blood flow continues unobstructed. The supporting peripheral wall is typically, but not exclusively, formed by a latticed support structure, which allows the stent to be inserted in a compressed state having a small external diameter up to the constricted point of the particular vessel to be treated and to be expanded thereafter with the aid of a balloon catheter, for example, enough that the vessel has the desired, enlarged internal diameter. To avoid unnecessary vascular damage, the stent only recoils elastically slightly or not at all after the expansion and after removal of the balloon, so that the stent only has to be expanded slightly beyond the desired final diameter during expansion. Further criteria which are desirable in regard to a stent include, for example, uniform area coverage and a structure which allows a certain flexibility in relation to the longitudinal axis of the stent. In practice, the stent is typically molded from a metallic material to implement the cited mechanical properties.

In addition to the mechanical properties of a stent, the stent is made with a biocompatible material to avoid rejection reactions. Currently, stents are used in approximately 70% of all percutaneous interventions; however, an in-stent restenosis occurs in 25% of all cases because of excess neointimal growth, which is caused by a strong proliferation of the arterial smooth muscle cells and a chronic inflammation reaction. Various approaches are used to reduce the restenosis rate.

In one approach for reducing the restenosis rate, a pharmaceutically active substance (active ingredient) is provided on the stent, which cataracts the mechanisms of restenosis and supports the course of healing. The active ingredient is applied in pure form or embedded in a carrier matrix as a coating or charged in cavities of the implant. Examples include, but are not limited to, the active ingredients sirolimus and paclitaxel.

A further promising approach for solving the problem is the use of biocorrodible metals and alloys because, typically, a permanent support function by the stent is not necessary; the initially damaged body tissue regenerates. Thus, it is suggested in German Patent Application No. 197 31 021 A1 that medical implants be molded from a metallic material whose main component is iron, zinc, and aluminum and/or is an element from the group consisting of alkali metals, alkaline earth metals. Alloys based on magnesium, iron, and zinc are described as especially suitable. Secondary components of the alloys may be from the group consisting of manganese, cobalt, nickel, chromium, copper, cadmium, lead, tin, thorium, zirconium, silver, gold, palladium, platinum, silicon, calcium, lithium, aluminum, zinc, and iron. Furthermore, the use of a biocorrodible magnesium alloy having a proportion of magnesium greater than 90%, yttrium 3.7-5.5%, rare earth metals 1.5-4.4%, and the remainder less than 1% is known from German Patent Application No. 102 53 634 A1, which is suitable, in particular, for producing an endoprosthesis, e.g., in the form of a self-expanding or balloon-expandable stent.

The use of biocorrodible metallic materials, in particular, but not limited to, biocorrodible magnesium or iron alloys in stents may result in a significant reduction of the restenosis rate. Notwithstanding the progress achieved, however, some problems are still to be solved; thus, the rise of the pH value caused by the corrosion of the material and the calcium flow into the surrounding cells thus induced may result in an undesired contraction of the vascular wall (vascular spasms).

### SUMMARY

The present disclosure provides several exemplary embodiments of the present invention.

One aspect of the present disclosure provides a stent, comprising (a) a biocorrodible metallic material, and (b) a coating or cavity filling comprising genistein.

Another aspect of the present disclosure provides a method of producing a stent, comprising (a) producing genistein as a coating material by a process comprising a biocorrodible metallic material, and a coating or cavity filling comprising genistein; and (b) forming a stent incorporating the genistein of step (a).

### DETAILED DESCRIPTION

A first exemplary embodiment provides an implant made of a biocorrodible metallic material with a coating or cavity filling which contains genistein.

Genistein (5,7-dihydroxy-3-(4-hydroxyphenyl)-4H-1-benzopyran-4-one, 4′,5,7-trihydroxy isoflavone, also called Differenol A, Prunetol, or Sophoricol) is a phytoestrogen from the group of isoflavones and a secondary metabolite from plants (Leguminosae, Papilionoidae, Rosaceae, among others; frequently in glycosylated form), but has also been found in cultures of microorganisms (Actinomycetes, Aspergillus, Mycobacteria, inter alia). Genistein has the following structural formula:

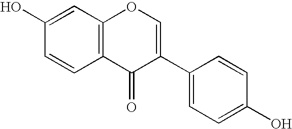

Genistein is taken in via food and may be detected in the serum of humans. The following pharmacological effects and action mechanisms are ascribed to genistein:
an agonist to the estrogen-β-receptor;
inhibitor of tyrosine kinases (inter alia, EGF-receptor tyrosine kinase);
inhibitor of topoisomerases (in particular, topoisomerase II);
inhibition of cardiac L-type Ca2+-channels; and
inhibition of NFkappa B.

In endothelial cells:
inhibition of the expression of the Vascular Endothelial Growth Factor (VEGF);
inhibition of collagen-induced platelet aggregation;
inhibition of the secretion of protein-1;
inhibition of the integrin-dependent leukocyte adhesion on endothelial cells;
inhibition of the adhesion of monocytes on endothelial cells after TNFα stimulation as a function of blood flow/physical forces;
inhibition of the expression of ACE in endothelial cells triggered by aldosterone; and
inhibition of the changes in the proteome of human endothelial cells, which have functions in metabolism, detoxification, and gene regulation, induced by homocysteine.

Effects on vascular smooth muscle cells:
inhibition of proliferation (in particular, via inhibition of tyrosine kinases);
inhibition of the migration induced by oxidative stress; and
inhibition of the endothelin-1 effect on the Ca2+-influx in smooth muscle cells of coronary arteries.

Furthermore, pharmacological effects of genistein on tumor cells and pathophysiological procedures in the postmenopausal period have been described.

Genistein has a higher potential for growth inhibition of human smooth muscle cells and for promoting growth of endothelial cells than the human estrogen 17β-estradiol. Furthermore, genistein, in addition to the specific biological effect on human smooth muscle cells/human endothelial cells, has the advantage, as an inhibitor of the proteins tyrosine kinase and topoisomerase II, that cardiac L-type Ca2+-channels are inhibited. Changes of the intracellular calcium concentration, which are more or less selectively controlled by the calcium channels, are decisive for many physiological processes. Inter alia, they result in synthesis and secretion of hormones, regulate the expression of genes, and control enzyme activities. So-called calcium channel blockers, in the form of genistein, for example, regulate the contractive force of the cardiac musculature and the smooth musculature of the vessels (typically reducing the contractive force of the cardiac musculature and the smooth musculature of the vessels), by inhibiting the calcium inflow into the muscle cells. This inhibition is especially advantageous for biocorrodible stents based on the metal alloys described hereinabove because an undesired contraction of the vascular wall (vascular spasms) may thus be avoided. Finally, initial experiments indicate that the presence of genistein supports the conversion of metal hydroxides initially occurring upon the corrosion, in particular, magnesium or iron hydroxide, into corresponding phosphates.

Genistein may be used in connection with biocorrodible metallic materials, in particular, but not limited to, magnesium or iron alloys, because the pH value connected with the corrosion of the active ingredient has no influence on the chemical stability of the molecule. Indications of metabolization of the active ingredient genistein induced by the pH value rise have not been found according to experiments of the applicant.

For purposes of the present disclosure, a coating is defined as at least partial application of the components to the main body of the stent. Preferably, the entire surface of the main body of the stent is covered by the coating. Alternatively, the genistein may be provided in a cavity of the stent.

The genistein is preferably, but not exclusively, embedded in a biodegradable organic carrier matrix. The biodegradable organic carrier matrix is preferably a polymer selected from the group consisting of polyglycolides, polylactides, polyhydroxy butyric acid, and poly-ε-caprolactone. The biodegradable organic carrier matrix is particularly poly-L-lactide.

Genistein is suitable, as a lipophilic active ingredient, in particular, for processing in biodegradable organic carrier matrices having hydrophobic character. The active ingredient is well soluble in organic solvents because of the lipophilic character. This makes it easier to incorporate the active ingredient into the polymer carrier matrix and supports a homogeneous and reproducible active ingredient distribution in the carrier matrix.

The coating or filling may contain, but is not limited to, the following additives:
Lipophilic vitamins (vitamins A, D, E, and K);
Fatty acids (such as, linoleic, oleic, palmitic, stearic, benzoic, cinnamic, linolenic, arachidonic, myristic, arachidic, behenic, palmitoleic, elaidic, vaccenic, icosenic, cetoleic, erucic, or nervonic acid);
Antioxidants (such as, alpha-tocopherol E 307, ascorbic acid E 300, ascorbyl palmitate E 304, butylhydroxytoluene (BHT) E 321, butylhydroxyanisol (BHA), calcium-disodium-EDTA E 385, calcium-L-ascorbate E 302, cal-cium hydrogen sulfite E 227, calcium sulfite E 226, citric acid E 330, delta-tocopherol E 309, diphosphate E 450, dodecyl gallate, lauryl gallate E 312, gamma-tocopherol E 308, isoascorbic acid E 315, potassium bisulfite E 228, potassium citrate E 332, potassium sulfite E 224, lecithin E 322, lactic acid E 270, sodium-L-ascorbate E 301, sodium-L-ascorbate E 301, sodium bisulfite E 222, sodium citrate E 331, sodium disulfite E 223, sodium isoascorbate E 316, sodium sulfite E 221, octyl gallate E 311, polyphosphate E 452, propyl gallate E 310, sulfur dioxide E 220, tocopherol E 306, triphosphate E 451, and tin-II-chloride E 512);
Emulsifiers (such as, ammonium phoshatide E 442, ascorbyl palmitate E 304, calcium phosphate E 341, calcium stearoyl-2-lactylate E 482, citric acid esters of monoglycerides and diglycerides of dietary fatty acids E 472c, diphosphate E 450, potassium phosphate E 340, lecithin E 322, sodium phosphate E 339, sodium stearoyl-2-lactylate E 481, phosphoric acid E 338, polyglycerin polyricinoleate E 476, polyoxyethylene (40) stearate E 431, polyphosphate E 452, polysorbate 20 E 432, polysorbate 40 E 434, polysorbate 60 E 435, polysorbate 65 E 436, polysorbate 80 E 433, propylene glycol alginate E 405, sorbitan monolaurate E 493, sorbitan monooleate E 494, sorbitan monopalmitate E 495, sorbitan monostearate E 491, sorbitan tristearate E 492, stearyl tartrate E 483, triphosphate E 451, and sugar glycerides E 474);
Phospholipids;
Fluorescent markers;
X-ray markers; and
Pigments.

For purposes of the present disclosure, biocorrodible refers to metallic materials in which degradation occurs in a physiological environment, which finally results in the entire implant or the part of the implant made of the material losing mechanical integrity. For purposes of the present disclosure, biocorrodible metallic materials particularly comprise metals and alloys selected from the group of elements consisting of iron, tungsten, and magnesium.

The biocorrodible material is preferably a magnesium or iron alloy. A biocorrodible magnesium alloy which contains yttrium and further rare earth metals is especially preferred because an alloy of this type is distinguished on the basis of its physiochemical properties and high biocompatibility, in particular, and of its degradation products.

A magnesium alloy of the composition of rare earth metals 5.2-9.9 weight-percent, yttrium 3.7-5.5 weight-percent, and the remainder less than 1 weight-percent is especially preferably used, magnesium making up the proportion of the alloy to 100 weight-percent. This magnesium alloy has already confirmed its special suitability experimentally and in initial clinical trials, i.e., the magnesium alloy displays a high biocompatibility, favorable processing properties, good mechanical characteristics, and corrosion behavior adequate for the intended uses. For purposes of the present disclosure, the collective term “rare earth metals” includes scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70), lutetium (71), and the like.

The composition of the metallic materials or magnesium or iron alloys is to be selected in such a way that they are biocorrodible. Artificial plasma, as has been previously described according to EN ISO 10993-15:2000 for biocorrosion assays (composition NaCl 6.8 g/l, CaCl₂ 0.2 g/l, KCl 0.4 g/l, MgSO4 0.1 g/l, NaHCO3 2.2 g/l, Na2HPO4 0.126 g/l, NaH2PO4 0.026 g/l), is used as a testing medium for testing the corrosion behavior of an alloy under consideration. For this purpose, a sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces. The artificial plasma according to EN ISO 10993-15:2000 corresponds to a medium similar to blood and thus represents a possibility for reproducibly simulating a physiological environment.

A second exemplary embodiment relates to the use of genistein as a coating material for a stent made of a biocorrodible material, in particular, a biocorrodible magnesium or iron alloy.

Stents made of the biocorrodible magnesium alloy WE43 (97 weight-percent magnesium, 4 weight-percent yttrium, 3 weight-percent rare earth metals besides yttrium) were coated as follows.

The magnesium surfaces of the stents were roughened by treatment with argon plasma to achieve greater adhesion of the active ingredient to the stent surface.

A 5×10-2 M methanolic solution of genistein was used and applied directly by spraying. The stents were crimped manually onto a balloon and dried at room temperature. Approximately 120 mg genistein was applied per stent.

For the release in porcine plasma, the stents were each transferred into a glass vial closable using a screw cap, admixed with 1 ml porcine plasma, closed, and shaken by machine at 37° C. in an incubator. At the predefined sample removal time, the stents were each removed and subjected to the elution procedure in a new vial having the corresponding volume of the fresh elution agent. Of the remaining plasma sample, 0.5 ml was admixed with 3 ml diethyl ether in a glass vial closable by a screw cap, shaken for 5 minutes, the plasma phase was frozen in the freezer, the diethyl ether was separated and evaporated until dried, and the residue was received with 0.5 ml mobile solvent (0.1 weight-percent aqueous orthophosphoric acid, acetonitrile (62/38)). The particular resulting solution was measured using HPLC.

The HPLC assays showed that the release of the active ingredient in porcine plasma was already terminated after approximately 10 minutes. Indications of instability of the active ingredient genistein, in connection with the contact of the genistein with the magnesium surface, were not recognized in the mass balances of the active ingredient or in the chromatograms.

Further assays on the release kinetics of genistein were performed from a polymer carrier matrix, more precisely poly-L-lactide. Genistein was used at a weight proportion of 30, 50, and 60 weight-percent in relation to the total weight of genistein and poly-L-lactide. The assays were performed in porcine blood plasma and performed analogously to the above-mentioned work steps without polymer matrix.

It has been shown that poly-L-lactide represents a suitable biodegradable carrier matrix for the release of genistein; and, in addition, a significant delay of the active ingredient release in relation to the application of the pure active ingredient may be achieved. Thus, the main quantity of the incorporated genistein is eluted from the poly-L-lactide carrier matrix after approximately 3.5 months.

All patents, patent applications and publications are incorporated by reference herein in their entirety.

What is claimed is:

1. A stent, comprising:
(a) a biocorrodible metallic material, and
(b) a coating or cavity filling comprising genistein.

2. The stent of claim 1, wherein the biocorrodible metallic material is an alloy of magnesium or iron.

3. The stent of claim 1, wherein the genistein is embedded in a biodegradable organic carrier matrix.

4. The stent of claim 3, wherein the biodegradable organic carrier matrix is a polymer selected from the group consisting of polyglycolides, polylactides, polyhydroxy butyric acid, and poly-ε-caprolactone.

5. The stent of claim 4, wherein the biodegradable organic carrier matrix is poly-L-lactide.

6. A method for producing a stent, comprising:
(a) providing a biocorrodible metallic material; and
(b) coating or cavity filling at least a portion of the biocorrodible metallic material with genistein.

7. The method of claim 6, wherein the biocorrodible metallic material is an alloy of magnesium or iron.

8. The method of claim 6, wherein the genistein is embedded in a biodegradable organic carrier matrix.

9. The method of claim 6, wherein the biodegradable organic carrier matrix is a polymer selected from the group consisting of polyglycolides, polylactides, polyhydroxy butyric acid, and poly-ε-caprolactone.

10. The method of claim 6, wherein the biodegradable organic carrier matrix is poly-L-lactide.
